(19) United States
(12) Patent Application Publication
(10) Pub. No.: US 2018/0325426 A1
(43) Pub. Date: Nov. 15, 2018

(54) Activities of Daily Living Monitoring and Reporting System

(71) Applicant: Algorthmic Intuition Inc., San Jose, CA (US)

(72) Inventors: Kevin A. Shaw, Millbrae, CA (US); Dan W. Brown, San Jose, CA (US)

(21) Appl. No.: 15/901,764

(22) Filed: Feb. 21, 2018

Related U.S. Application Data
(60) Provisional application No. 62/590,140, filed on Nov. 22, 2017.
(60) Provisional application No. 62/505,784, filed on May 12, 2017.

(51) Int. Cl. A61B 5/11; G06F 11/34; G06F 11/30; G06N 3/08; G01P 13/00; A61B 5/00

(52) U.S. Cl. CPC A61B 5/1118; G06F 11/3438; G06F 11/3013; G01C 19/00; G01P 13/00; A61B 5/6823; A61B 5/6833; G06N 3/08

(57) ABSTRACT

A user-wearable electronic device includes a housing configured to be worn on a user's torso, a plurality of sensors disposed in the housing, including a first sensor to sense motion of the user and produce raw activities of daily living (ADL) data, and a biometric sensor to sense one or more biometric characteristics of the user. One or more processors in the electronic device or an intermediary device generate, for a sequence of time periods, ADL identification information by processing the raw ADL data using one or more neural networks pre-trained to recognize a predefined set of ADLs. Each pre-trained neural network includes a plurality of neural network layers, including at least one layer that includes a recurrent neural network. Reports that include ADL information corresponding to the generated ADL identification information for time periods in the sequence of time periods are transmitted to a monitoring system.

### RELATED APPLICATIONS

This application claims priority to U.S. Provisional Patent Application 62/590,140, filed Nov. 22, 2017, and to Provisional Patent Application 62/505,784, filed May 12, 2017, each of which is hereby incorporated by reference in its entirety.

### TECHNICAL FIELD

This relates generally to health monitoring devices, including but not limited to wearable electronic wellness tracking devices that monitor and report activities of daily living.

### BACKGROUND

Activities of daily living (ADL) are routine activities that people tend to do every day, and a person's ability to perform them is important in determining the person's ability to live independently. The level of assistance a person may need with regard to day-to-day living can be determined by monitoring the ADLs the person performs over a given period of time.

Healthcare professionals have an interest in monitoring ADLs in order to evaluate their patients, but conventional ADL monitoring systems can be burdensome and expensive, requiring multiple monitoring devices. Further, conventional ADL monitoring systems can be inaccurate due to wide ranges of motions associated with each ADL, which vary from person to person. Additionally, conventional ADL monitoring systems can lack the flexibility and mobility required for tracking a person in multiple locations around a house, due to rigid vision systems that are limited in terms of lighting and field of view. Camera-based ADL monitoring systems are also associated with privacy issues, as people dislike the idea of being on camera while performing certain personal activities like bathing and toileting.

### SUMMARY

Accordingly, there is a need for ADL monitoring and reporting systems with improved monitoring methods and more portable and self-contained devices. Such methods and devices optionally complement or replace conventional methods and devices for monitoring and reporting ADLs. Such methods provide more accurate ADL classifications by using pre-trained neural networks to interpret raw data, and such devices eliminate the need to install multiple sensing components by being self-contained in a wearable form factor, thereby creating more accurate results with less burdensome hardware. The aforementioned deficiencies and other problems associated with ADL monitoring systems are reduced or eliminated by the disclosed ADL monitoring and reporting systems.

In accordance with some embodiments, a user-wearable electronic device includes a housing configured to be worn on a user's torso, the housing including an interface configured to be in direct contact with the user's skin; a plurality of sensors disposed in the housing, including a first sensor to produce raw ADL data, and a biometric sensor coupled to the interface and configured to sense one or more biometric characteristics of the user and generate corresponding biometric data. The device further includes one or more processors, disposed in the housing and coupled to the one or more sensors, and configured to generate, for each time period in a sequence of successive time periods, ADL identification information for the time period by processing the raw ADL data produced by the first sensor using one or more neural networks pre-trained to recognize a predefined set of ADLs. In some embodiments, at least one of the pre-trained neural networks includes a plurality of neural network layers, at least one layer of the plurality of neural network layers comprising a recurrent neural network, wherein an output of the one or more neural networks for each time period corresponds to the generated ADL identification information for the time period. In some embodiments, each pre-trained neural network includes a plurality of neural network layers, at least one layer of the plurality of neural network layers comprising a recurrent neural network.

The device also includes a transmitter, disposed in the housing and coupled to at least one processor of the one or more processors, to transmit one or more reports corresponding to the user, wherein a respective report for the user includes ADL information corresponding to the generated ADL identification information for one or more time periods in the sequence of time periods.

In some other embodiments, obtaining raw ADL data corresponding to a user and processing the raw ADL data to produce ADL identification information for one or more time periods in a sequence of successive time period is distributed over two or more devices, at least one of which processes the raw ADL data, or related ADL information, using one or more neural networks pre-trained to recognize a predefined set of ADLs. For example, in some embodiments, a user-wearable electronic device includes a housing configured to be worn by or embedded in a device worn by a user; one or more sensors disposed in the housing, including a first sensor to sense motion of the user and produce raw ADL data corresponding to the user. The device also includes a transmitter, optionally disposed in the housing, to transmit the ADL data or ADL information generated from the ADL data, to a monitoring system or to an intermediate device at which the ADL data or ADL information is further processed to generate, for each time period in a sequence of successive time periods, ADL identification information for the time period by processing the raw ADL data produced by the first sensor or ADL information generated from the ADL data, using one or more neural networks pre-trained to recognize a predefined set of ADLs. In some of these embodiments, at least one of the pre-trained neural networks includes a plurality of neural network layers, at least one layer of the plurality of neural network layers comprising a recurrent neural network, wherein an output of the one or more neural networks for each time period corresponds to the generated ADL identification information for the time period. In some of these embodiments, each of the pre-trained neural networks includes a plurality of neural network layers, at least one layer of the plurality of neural network layers comprising a recurrent neural network.

### BRIEF DESCRIPTION OF THE DRAWINGS

For a better understanding of the various described embodiments, reference should be made to the Description of Embodiments below, in conjunction with the following drawings in which like reference numerals refer to corresponding parts throughout the figures.

FIG. 1A is a context diagram illustrating a user-wearable electronic device, configured to perform ADL monitoring and reporting, in accordance with some embodiments.

FIG. 1B is a context diagram illustrating an example layout of a living environment including beacons in accordance with some embodiments.

FIG. 2 is a block diagram illustrating components of a user-wearable electronic device in accordance with some embodiments.

FIGS. 3A-3B are block diagrams illustrating a user-wearable electronic device and an intermediary device in accordance with a first set of embodiments.

FIGS. 3C-3D are block diagrams illustrating a user-wearable electronic device and an intermediary device in accordance with a second set of embodiments.

FIG. 4 is a block diagram illustrating an implementation of a monitoring system in accordance with some embodiments.

FIG. 5 is a block diagram illustrating an implementation of a mobile device in accordance with some embodiments.

FIG. 6A is a block diagram illustrating an implementation of a user profile database in accordance with some embodiments.

FIG. 6B is a block diagram illustrating an implementation of a user ADL database in accordance with some embodiments.

FIG. 6C is a block diagram illustrating information included in a user report and information included in a raw data report, in accordance with some embodiments

FIG. 6D is a block diagram illustrating neural network configurations for particular ability categories in accordance with some embodiments.

FIG. 7 is a flow chart illustrating data flow in a user-wearable electronic device, configured to perform ADL monitoring and reporting, in accordance with some embodiments.

FIGS. 8A-8F illustrate an embodiment of a user-wearable electronic device from multiple perspectives.

FIGS. 9A-9B illustrate a set of reports displayed in user interfaces of an ADL monitoring system in accordance with some embodiments.

### DESCRIPTION OF EMBODIMENTS

Reference will now be made in detail to embodiments, examples of which are illustrated in the accompanying drawings. In the following detailed description, numerous specific details are set forth in order to provide a thorough understanding of the various described embodiments. However, it will be apparent to one of ordinary skill in the art that the various described embodiments may be practiced without these specific details. In other instances, well-known methods, procedures, components, circuits, and networks have not been described in detail so as not to unnecessarily obscure aspects of the embodiments.

It will also be understood that, although the terms first, second, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact, unless the context clearly indicates otherwise.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

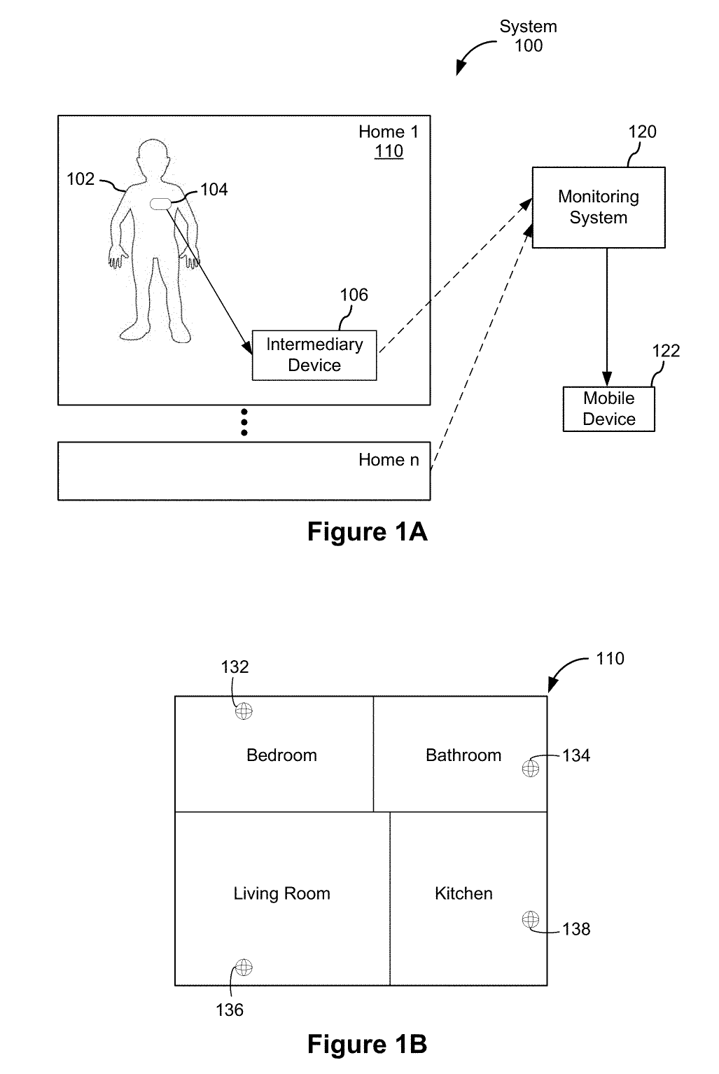

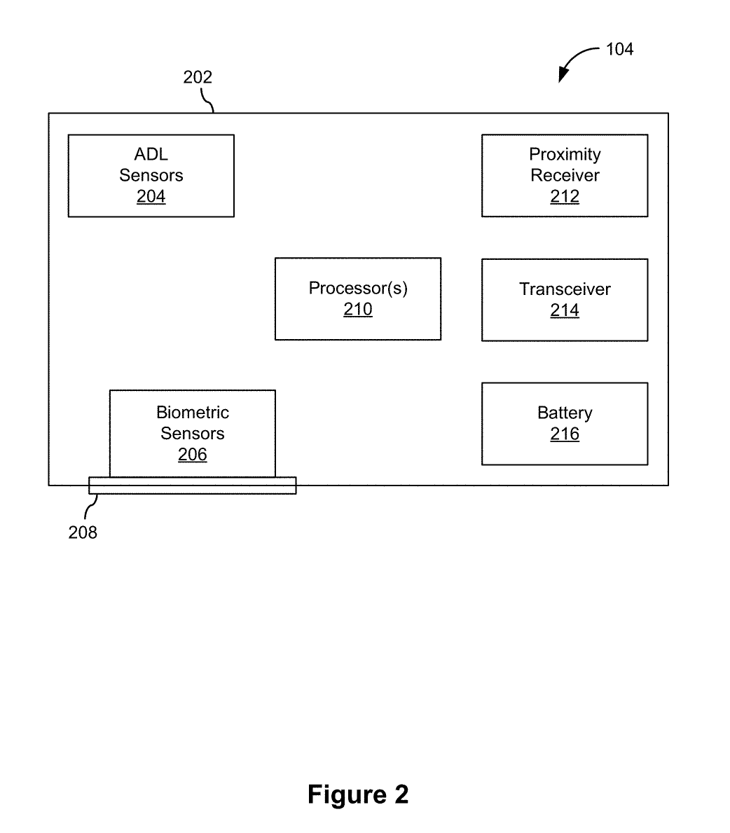

Attention is now directed toward embodiments of activities of daily living (ADL) monitoring and reporting systems in accordance with some embodiments. FIG. 1A is a block diagram illustrating a sample embodiment of an ADL monitoring and reporting system 100. Person 102, sometimes herein called a user, is monitored by user-wearable ADL monitoring device 104. In some embodiments, ADL monitoring device 104 is affixed directly to the person's skin. Optionally, ADL monitoring device 104 is physically coupled to person 102 by way of clothing or any other object that is attached to the person. In some embodiments, ADL monitoring device 104 reports ADL data, or ADL identification information generated from the ADL data, to intermediary device 106, which receives the ADL data or ADL identification information and transmits a subset of the data, all of the data, or a representation of the data to monitoring system 120, sometimes herein called a monitoring station. In some embodiments, intermediate device 106 processes ADL data received from ADL monitoring device 104, for example using one or more neural networks, as discussed in more detail below, to produce ADL identification information, and transmits the ADL identification information to a monitoring system, from which authorized users can access the ADL identification information. In some other embodiments, ADL monitoring device 104 directly transmits ADL data or ADL identification information to monitoring system 120.

In some embodiments, person 102, ADL monitoring device 104, and intermediary device 106 are located in a home 110. It is understood that home 110 is any living arrangement in which a healthcare professional or anyone in a caregiver role may wish to monitor ADLs of one or more persons, including, for example, a house, an apartment, an assisted care facility, or a hospital. In some embodiments, home 110 is one of a plurality of homes, such as n homes where n is an integer greater than 1, or greater than 2. Each home 110 typically houses one or more people and one or more ADL monitoring devices, all of which report ADL data (e.g., ADL identification information) to monitoring system 120, either directly or through one or more intermediary devices 106. In other embodiments, home 110 is the only home from which ADL data is reported to monitoring system 120.

In some embodiments, mobile device 122 is communicatively coupled to monitoring system 120, and provides access to ADL reports for healthcare professionals (or those otherwise fulfilling a caregiver role) wishing to monitor ADL data from one or more persons 102. In embodiments in which the ADLs of multiple people are being monitored, mobile device 122 optionally provides access to a desired subset of the people whose ADL's are being monitored. For example, a caregiver is optionally given access, via mobile device 122, to the ADL information for a particular subset of persons whose ADL information is being reported to monitoring system 120. Access rights are optionally assigned according to security levels, relevance levels, legal constraints, and/or on a need-to-know basis. For example, a first person's caregiver may be given access to ADL for the first person, while a second person's caregiver may be given access to ADL reports for the second person. As another example, for embodiments in which monitoring system 120 receives ADL reports from a plurality of different homes or rooms in a nursing home, caregivers may only have access to ADL reports for users assigned to each respective caregiver. In yet another example, different caregivers are given access to ADL information at different levels of granularity. For example, relatives of a monitored person may be given access only to summary reports for the monitored person, for example daily summary reports, without access to more detailed ADL information, while healthcare professionals assigned to monitor the person have access all ADL information or more detailed ADL information. Optionally or alternatively, for embodiments in which there is no mobile device 122, monitoring system 120 provides access to ADL reports.

FIG. 1B illustrates an example layout of a home 110, which includes four rooms in this example. In some embodiments, each room of a plurality of rooms of a home include a location or proximity beacon 132-138. In some embodiments, only a subset of the rooms in a home include a location or proximity sensor 132-138. The illustrated placement of beacons 132-138 in FIG. 1B is merely an example, and it is understood that placement of each beacon 132-138 in each respective room may depend on factors such as room dimensions, contents, activity types, safety constraints, and the like. Operation of location or proximity beacons 132-138 is discussed below with reference to FIG. 2.

FIG. 2 is a block diagram illustrating components of the user-wearable electronic device 104 (see FIG. 1A), in accordance with some embodiments. User-wearable electronic device 104 includes housing 202, one or more ADL sensors 204, one or more biometric sensors 206, interface 208 for coupling the one or more biometric sensors 206 to a user's skin, configured to be in direct contact with a user's skin, one or more processors 210, proximity receiver 212 (sometimes called a location or proximity sensor), transceiver 214, and battery 216. Transceiver 214 typically includes a transmitter and receiver, with the transmitter being used to transmit user reports that include ADL information regarding the user wearing the device, and the receiver being used to receive software and configuration updates, and optionally commands and other information. Battery 216 is typically a rechargeable battery, implemented using any appropriate battery technology. In some embodiments, device 104 optionally includes only a subset of the aforementioned components. For example, in some embodiments, user-wearable electronic device 104 may include an ADL sensor 204 but no biometric sensor 206 and interface 208. In some embodiments, user-wearable electronic device 104 does not include proximity receiver 212.

In some embodiments, housing 202 is configured to be worn on or near a user's torso. In some embodiments, housing 202 is configured to be in direct contact with the user's skin, or affixed to an object, such as an adhesive layer, that is in direct contact with the user's skin. In some embodiments, housing 202 is placed on or near any portion of the user's torso that moves with the user, such as the chest, stomach, back, shoulder, or side of the body. In some embodiments, housing 202 has a compact form factor that allows device 104 to be worn on the user's body without causing a nuisance to the user. For example, housing 202 may have a length no greater than 10 centimeters (cm), a width no greater than 6.5 cm, and a thickness no greater than 0.5 cm. A typical size is 5 to 8.5 cm in length, 2.5 to 5 cm in width, and 0.1 to 0.25 cm in thickness. Other dimensions are possible as well, with a person of ordinary skill in the art recognizing that the bigger the housing, the more of a nuisance its presence may be on the user's body. However, since a bigger housing can fit more components and/or a larger battery 216, and different dimensions can be optimized to fit various sizes of internal components, the exact dimensions of housing 202 are not meant to be limiting to any of the disclosed embodiments. In some embodiments, the form factor of housing 202 is configured to allow device 104 to fit within underclothing, such as a band of an article of underclothing, which helps to ensure the device is maintained in contact with the user's skin. Optionally, housing 202 is configured to allow user-wearable electronic device 104 to be integrated into an article of clothing that conforms to the skin. Additionally, housing 202 includes a waterproof or water-resistant seal so that user-wearable electronic device 104 is washable with a machine washer and can withstand activities involving water (e.g., bathing) and environments having high humidity. In order to further minimize any discomfort that may be caused by the presence of housing 202 on the user's body, in some embodiments, housing 202 and all components within the housing may be configured to have a total weight no greater than 60 grams. In other embodiments, the total weight may be no greater than 25 grams, 20 grams, 15 grams, or 10 grams.

In some embodiments, ADL sensors 204 include an accelerometer, an orientation sensor, a motion sensor, a gyroscopic sensor, or a combination thereof. In some embodiments, ADL sensors 204 include only one of the aforementioned sensors. ADL sensors 204 generate acceleration data, orientation data, motion data, gyroscopic data, or a combination thereof in response to movements associated with ADLs. In some embodiments, a predefined set of basic ADLs monitored by user-wearable electronic device 104 includes dressing, eating, transferring/ambulation, continence/toileting, bathing/hygiene, sitting, and sleeping/napping. Optionally, a predefined set of instrumental ADLs monitored by user-wearable electronic device 104 includes housekeeping, meal preparation, transportation, talking/socialization/communication, managing personal finances/accounting, and managing medications, or any subset of those activities. In various embodiments, user-wearable electronic device 104 is configured to monitor a subset of p basic ADLs, where p is 3, 4 or 5, or more generally p is an integer greater than 2, greater than 3, or greater than 4.

In some embodiments, biometric sensors 206 include a temperature sensor for sensing temperature of the user, an electrocardiography (EKG) sensor for detecting electrical activity associated with the user's heart, a heart rate sensor for measuring the user's heart rate, a blood pressure sensor for measuring at least one parameter of the user's blood pressure, a bioimpedance sensor for measuring at least one parameter of the user's body composition, a total water content sensor, a photoplethysmograph (PPG) sensor for measuring the user's heart rate, or any subset or combination thereof. In some embodiments, biometric sensor 206 includes only one of the aforementioned sensors (e.g., a heart rate sensor), or only two of the aforementioned sensors (e.g., a temperature sensor and heart rate sensor). In some embodiments, biometric sensor 206 is coupled to interface 208 of housing 202 to allow biometric sensor 206 close access to the user's skin. In some embodiments, interface 208 is an opening in housing 202, which allows for direct physical contact of biometric sensor 206 with the user's skin. In other embodiments, interface 208 is a region of housing 202 in which a cross section of housing material is smaller (i.e., thinner) when compared to a region outside of interface 208, which is useful for establishing electrical contact with the user's skin. By enabling direct or close access to the user's skin, interface 208 allows biometric sensor 206 to accurately sense biometric characteristics of the user.

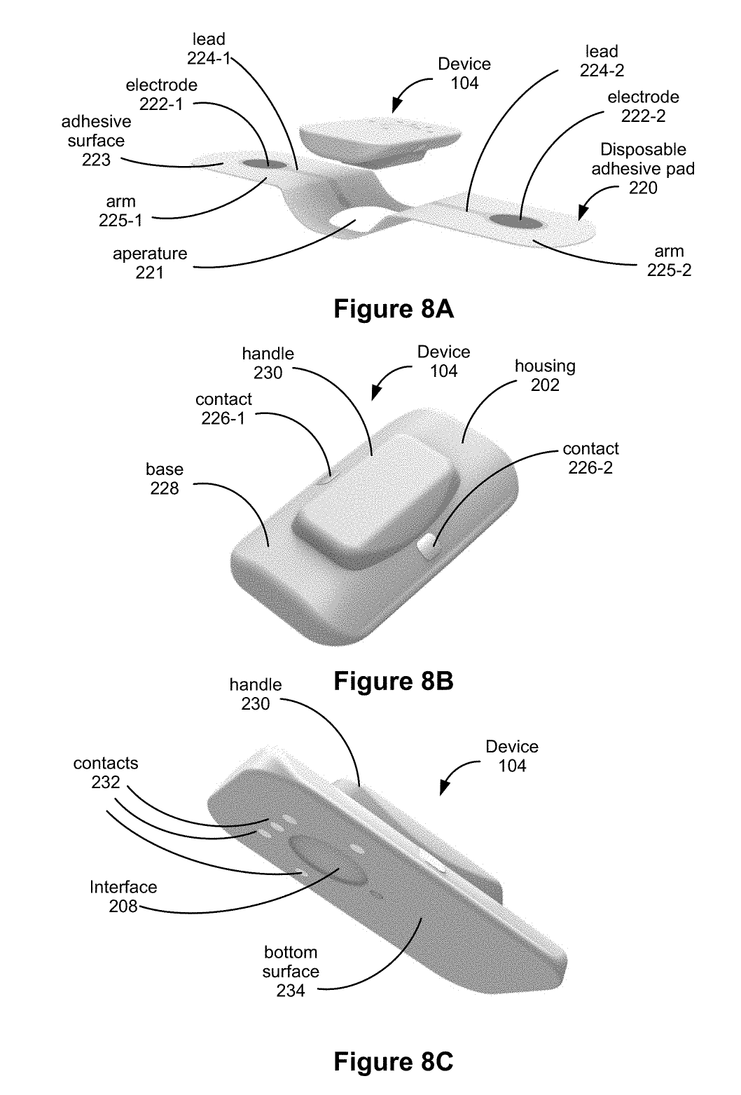

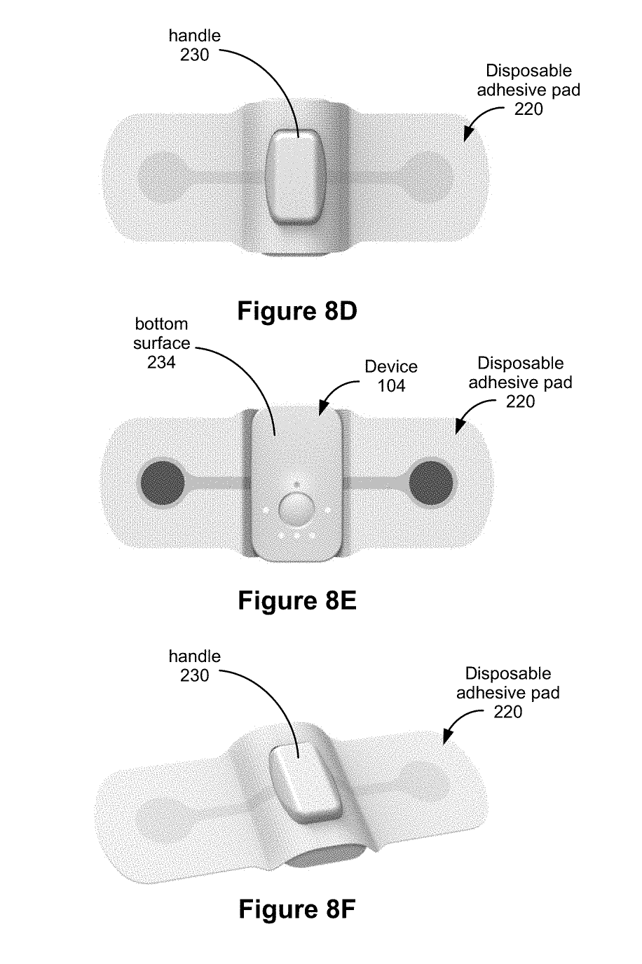

FIGS. 8A-8F illustrate an embodiment of a user-wearable electronic device 104 from multiple perspectives. FIG. 8A shows an exploded, bottom perspective view of device 104 in conjunction with a disposable adhesive pad 220, while FIG. 8B shows a top perspective view of device 104 and FIG. 8C shows a different bottom perspective view of device 104 than FIG. 8A. Referring to FIGS. 8A, 8B, and 8C, in some embodiments, a handle portion 230 (see FIG. 8B) of device 104's housing 202 is configured to mate with or protrude through an aperture 221 in disposable adhesive pad 220. In some embodiments, disposable adhesive pad 220 includes electrodes 222-1 and 222-2 that are connected to a biometric sensor 206 inside housing 202 via leads 224-1 and 224-2 on a surface of disposable adhesive pad 220, and contacts 226-1 and 226-2 on the exterior of housing 202 (see FIG. 8B). In some embodiments, housing 202 of device 140 includes a base 228 and a handle 230, which protrudes from base 228 and is configured to mate with or protrude through aperture 221 of disposable adhesive pad 220. In some embodiments, disposable adhesive pad 220 includes arms 225-1, 225-2 that extended away from device 104 when device 104 is mated with disposable adhesive pad 220, and each arm 225 includes an adhesive surface 223 for holding electrodes 222 and a bottom surface 234 of device 104 against the skin of a user.

In some embodiments, bottom surface 234 of device 234 includes a plurality of contacts 232 for making electrical and mechanical contact with a user's skin, and providing electrical signals to one or more biometric sensors 206 inside housing 202. In some embodiments, bottom surface 234 of device 234 includes interface 208 through which one or more biometric sensors 206 make contact with or are coupled to the skin of the user.

FIG. 8D shows a top view of device 104 inserted into disposable adhesive pad 220, while FIG. 8E shows a bottom view of device 104 inserted into disposable adhesive pad 220 and FIG. 8F shows a top perspective view of device 104 inserted into disposable adhesive pad 220.

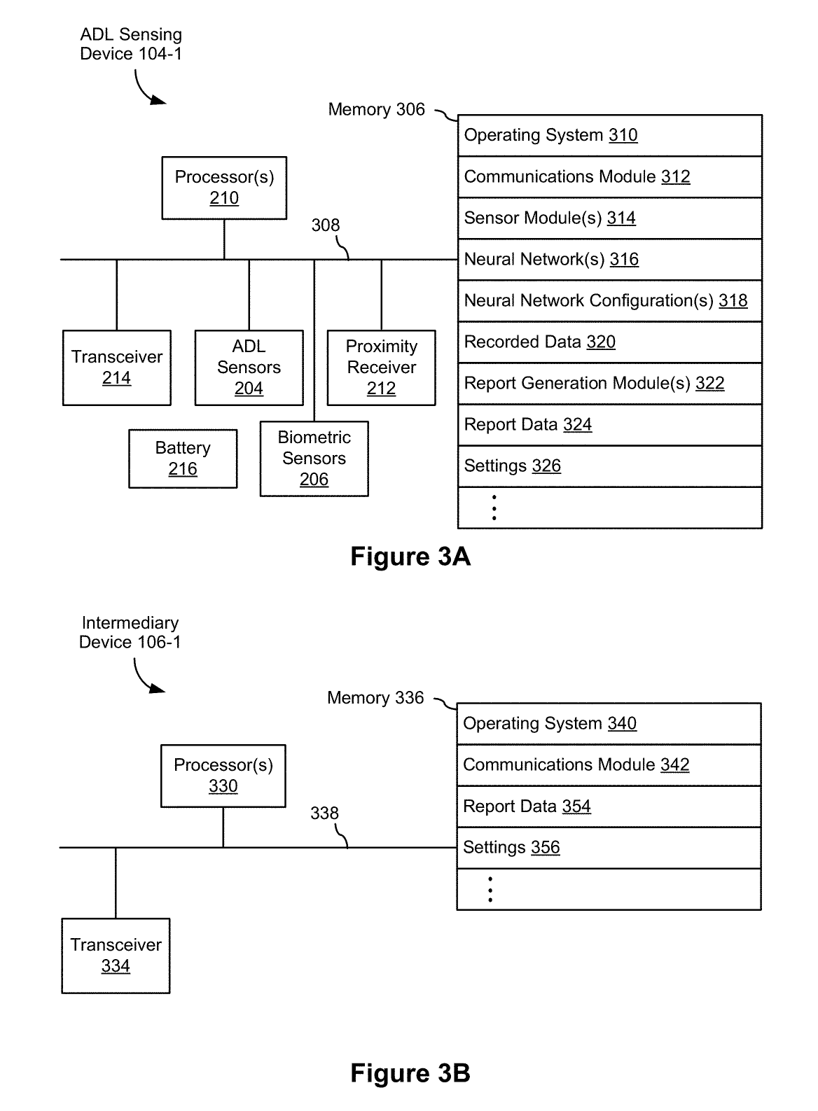

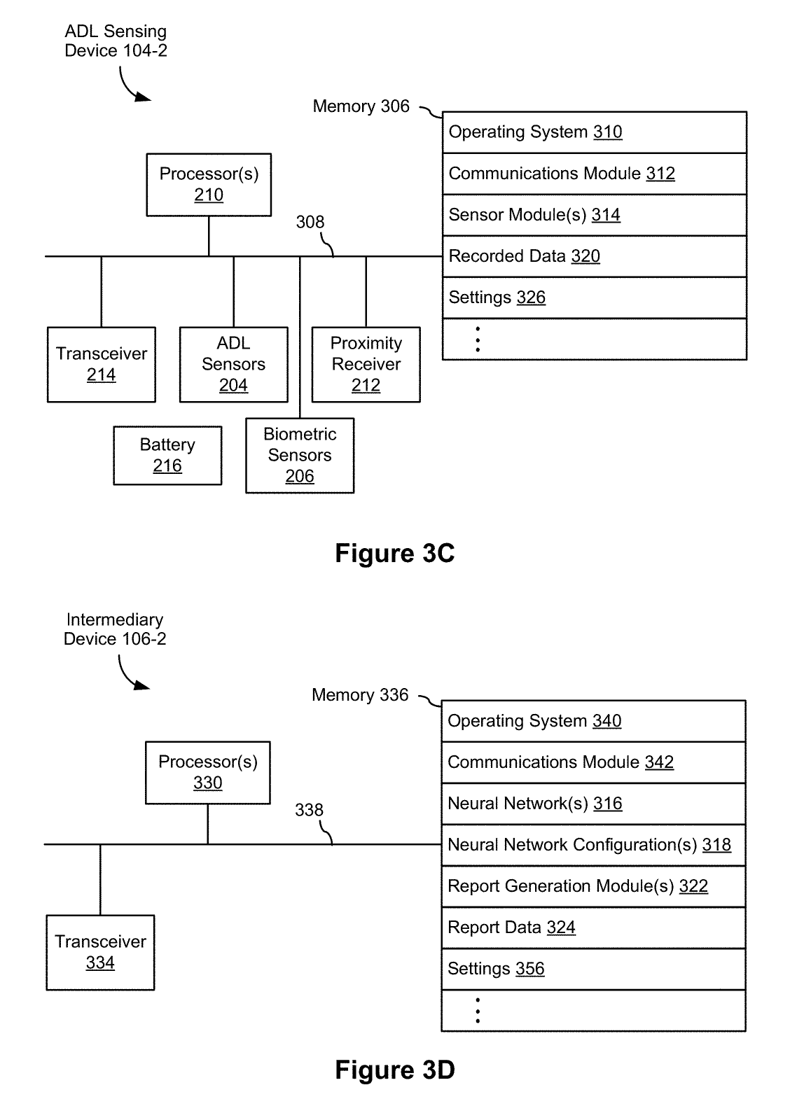

FIG. 3A is a block diagram illustrating a user-wearable electronic device 104-1 in accordance with some embodiments. In some embodiments, device 104-1 includes one or more processors 210, sometimes called CPUs, or hardware processors, or microcontrollers; transceiver 214; one or more biometric sensors 206; ADL sensors 204; memory 306; and one or more communication buses 308 for interconnecting these components. Device 104-1 optionally includes proximity receiver 212. For example, if the system 100 determines a user's ADLs in part based on the proximity of the user to one or more beacons, then device 104-1 includes a proximity receiver 212. Communication buses 308 optionally include circuitry (sometimes called a chipset) that interconnects and controls communications between system components.

As explained above with reference to FIG. 2, device 104-1 includes a battery 216. The inclusion of battery 216 in device 104-1 enables operation of device 104-1 as a mobile device, without connection to an external power source (i.e., external to device 104-1). In some embodiments, battery 216 is sized, or has sufficient capacity, to enable operation of device 104-1 for at least one day, or at least two days, or three days, or a week, before the battery requires recharging.

Memory 306 includes high-speed random access memory, such as DRAM, SRAM, DDR RAM or other random access solid state memory devices, and may include non-volatile memory, such as flash memory devices, or other non-volatile solid state storage devices. Memory 306, or alternately the non-volatile memory device(s) within memory 306, comprises a non-transitory computer readable storage medium. In some embodiments, memory 306, or the computer readable storage medium of memory 306 stores the following programs, modules, and data structures, or a subset or superset thereof:
operating system 310, for handling basic computer functions of device 104-1;
communications module 312 used for transmitting ADL data and/or reports via transceiver 214 to monitoring system 120, or an intermediary device 106, or other system; and optionally for receiving updates, instructions and/or other information from one or more external sources, such as monitoring system 120, via transceiver 214;
sensor module(s) 314, sometimes implemented as device drivers, for controlling and/or receiving data from ADL sensors 204, biometric sensors 206, and proximity receiver 212;
one or more neural networks 316, described in more detail elsewhere in this document, for processing raw data from at least one of the ADL sensors 204 so as to determine which activities of daily living the user of the device has been engaged in during each of a sequence of time periods;
neural network configuration(s) 318, which are used to configure the one or more neural networks; the neural network configuration(s) 318 are generated by one or more other systems configured to training one or more similar neural networks using training data, and then included in device 104-1, or transmitted to device 104-1; when the one or more neural networks 316 are configured using neural network configuration(s) 318, the one or more neural networks 316 are pre-trained neural networks;
recorded data 320, which includes recorded raw ADL data from the ADL sensors 204, and/or biometric sensors 206, and optionally proximity information associated with the information received from proximity receiver 212;
one or more report generation modules 322, which generate reports, described in more detail below, for transmission to an intermediary device, a monitoring system, or other system from which ADL information regarding the user is retrieved;
report data 324, which are the aforementioned reports, or data included in those reports; and
settings 326, typically including a device identifier, optionally including an identifier of the user of the device 104-1, and optionally including settings that enable and disable various features of device 104-1.

Each of the above identified elements may be stored in one or more of the previously mentioned memory devices that together form memory 306. Each of the above mentioned modules or programs, including the aforementioned modules and operating system, corresponds to a set of instructions and data for performing a function described above. The above identified modules or programs (i.e., sets of instructions) need not be implemented as separate software programs, procedures or modules, and thus various subsets of these modules may be combined or otherwise re-arranged in various embodiments. In some embodiments, memory 306 may store a subset of the modules and data structures identified above. Furthermore, memory 306 may store additional modules and data structures not described above. In some embodiments, the programs, modules, and data structures stored in memory 306, or the computer readable storage medium of memory 306, provide instructions for implementing respective operations of the methods described herein.

Although FIG. 3A shows an electronic device 104-1, FIG. 3A is intended more as a functional description of the various features which may be present in a user-wearable electronic device, than as a structural schematic of the embodiments described herein. In practice, and as recognized by those of ordinary skill in the art, items shown separately could be combined and some items could be separated.

FIG. 3B is a block diagram illustrating an intermediary device 106-1 in accordance with some embodiments. In some embodiments, device 106-1 includes one or more processors 330, sometimes called CPUs, or hardware processors, or microcontrollers; transceiver 334; memory 336; and one or more communication buses 338 for interconnecting these components. Communication buses 338 optionally include circuitry (sometimes called a chipset) that interconnects and controls communications between system components.

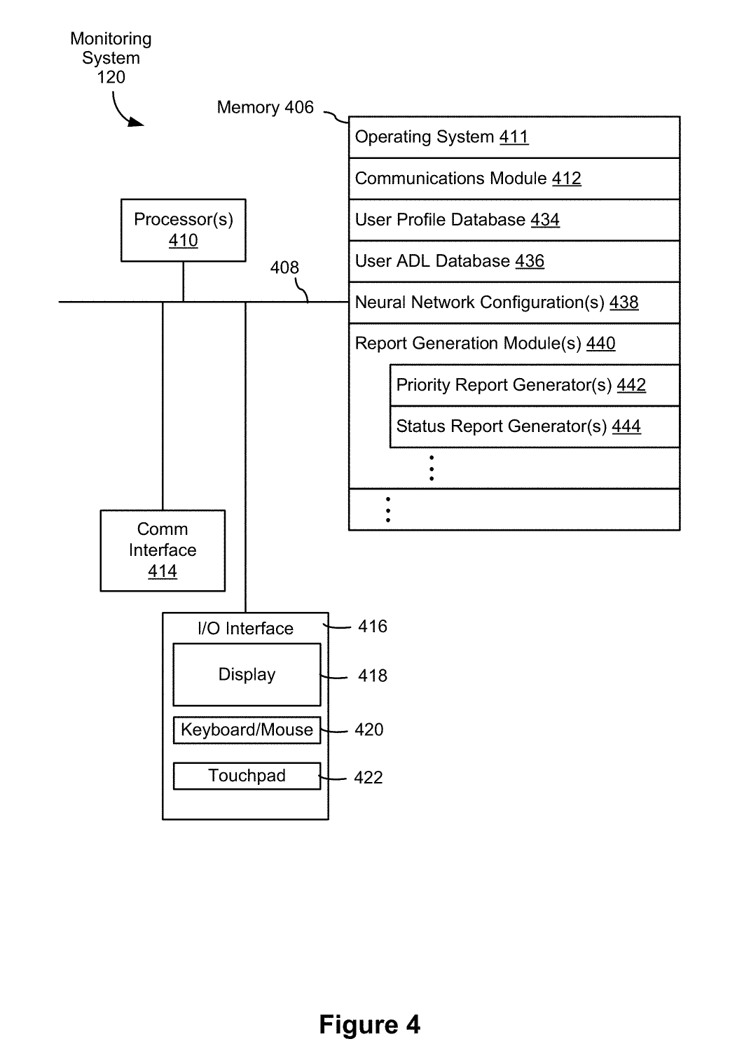

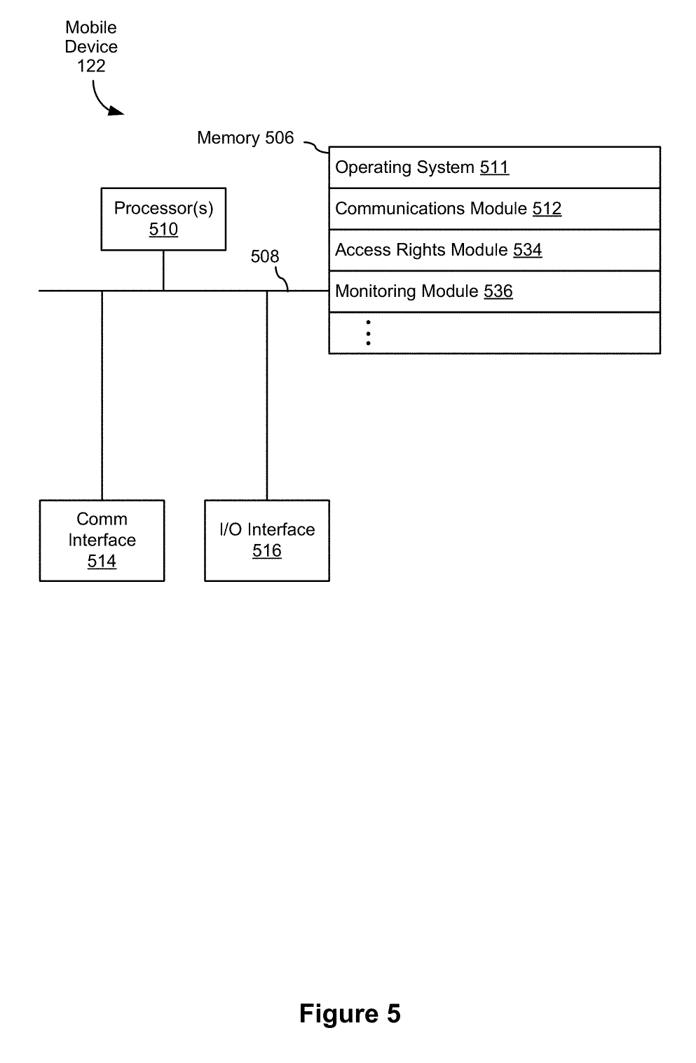

Memory 336 includes high-speed random access memory, such as DRAM, SRAM, DDR RAM or other random access solid state memory devices, and may include non-volatile memory, such as flash memory devices, or other non-volatile solid state storage devices. Memory 336, or alternately the non-volatile memory device(s) within memory 336, comprises a non-transitory computer readable storage medium. In some embodiments, memory 336, or the computer readable storage medium of memory 336 stores the following programs, modules, and data structures, or a subset or superset thereof:
operating system 340, for handling basic computer functions of device 106-1;
communications module 342 used for receiving reports from device 104-1 via transceiver 334; for receiving updates, instructions and optionally other information from monitoring system 120 or other system (i.e., other than intermediary device 106-1 and ADL sensing device 104-1) via transceiver 334; and for forwarding or sending reports or data to monitoring system 120 via transceiver 334;
report data 354, which are reports received from device 104-1, or data included in those reports, which are stored for transmission to a monitoring system (such as system 120, FIG. 4), or other system (such as system 122, FIG. 5) from which ADL information regarding the user is retrieved by a caregiver; and
settings 356, typically including a device identifier, and optionally including settings that enable and disable various features of device 106-1.

Each of the above identified elements may be stored in one or more of the previously mentioned memory devices that together form memory 336. Each of the above mentioned modules or programs, including the aforementioned modules and operating system, corresponds to a set of instructions and data for performing a function described above. The above identified modules or programs (i.e., sets of instructions) need not be implemented as separate software programs, procedures or modules, and thus various subsets of these modules may be combined or otherwise re-arranged in various embodiments. In some embodiments, memory 336 may store a subset of the modules and data structures identified above. Furthermore, memory 336 may store additional modules and data structures not described above. In some embodiments, the programs, modules, and data structures stored in memory 336, or the computer readable storage medium of memory 336, provide instructions for implementing respective operations of the methods described herein.

Although FIG. 3B shows an electronic device 106-1, FIG. 3B is intended more as a functional description of the various features which may be present in an intermediary electronic device, than as a structural schematic of the embodiments described herein. In practice, and as recognized by those of ordinary skill in the art, items shown separately could be combined and some items could be separated.

In embodiments represented by FIGS. 3A and 3B, ADL sensing device 104-1 processes raw ADL data 320, obtained from one or more ADL sensors 204, using one or more neural networks 316 configured by neural network configuration(s) 318, and generates reports 324 using report generation module(s) 322 for transmission to an intermediary device (e.g., intermediary device 106-1), a monitoring system (e.g., monitoring system 120), or other system from which ADL information regarding the user is retrieved by a caregiver.

FIGS. 3C and 3D show another ADL sensing device 104-2 and intermediary device 106-2 in accordance with some embodiments. Features shared with FIGS. 3A and 3B are similarly numbered, and some are not further discussed for purposes of brevity. In these embodiments, the neural network processing modules (316, 318) and report generation module(s) 322 are located (e.g., in memory 336) in intermediary device 106-2. In these embodiments, ADL sensing device 104-2 transmits raw ADL data (e.g., recorded data 320), or data that has been initially or slightly processed to intermediary device 106-2, and intermediary device 106-2 processes the raw ADL data (or slightly processed data) received from ADL sensing device 104-2, using neural networks 316, configured using neural network configuration(s) 318 as described above, and generates reports or report data 324 for monitoring system 120, or other system from which ADL information regarding the user is retrieved by a caregiver, using one or more report generation modules 322 as described above.

Each of the above identified elements may be stored in one or more of the previously mentioned memory devices that together form memory 306 (of ADL sensing device 104-2) and/or memory 336 (of intermediary device 106-2). Each of the above mentioned modules or programs, including the aforementioned modules and operating system, corresponds to a set of instructions and data for performing a function described above. The above identified modules or programs (i.e., sets of instructions) need not be implemented as separate software programs, procedures or modules, and thus various subsets of these modules may be combined or otherwise re-arranged in various embodiments. In some embodiments, memory 306 and/or memory 336 may store a subset of the modules and data structures identified above. Furthermore, memory 306 and/or memory 336 may store additional modules and data structures not described above. In some embodiments, the programs, modules, and data structures stored in memory 306 and/or memory 336, or the computer readable storage medium of memory 306 and/or memory 336, provide instructions for implementing respective operations of the methods described herein.

As recognized by those of ordinary skill in the art, embodiments corresponding to FIGS. 3A-3B and embodiments corresponding to FIGS. 3C-3D are merely two nonlimiting examples of distributed processing embodiments suitable for generating and processing raw ADL data so as to produce reports or report data having ADL identification information for a respective user. Stated another way, at least a portion of the neural network processing, at least a portion of the report generation processing, and/or other processing functions described herein are performed solely by processor(s) 210 of the ADL sensing device 104 in some embodiments, performed solely by processor(s) 330 of the intermediary device 106 in some embodiments, or performed by a combination of processors 210 and 330 in some embodiments.

In some embodiments, ADL sensing device 104 carries out all of the processing and transmits reports directly to a monitoring system, or other system from which ADL information regarding the user is retrieved by a caregiver, rendering an intermediary device unnecessary. In some embodiments, ADL sensing device 104 carries out all of the neural network processing and computations in real time, but only periodically sends ADL reports (e.g., once an hour, once every four hours, or once every eight hours), or only sends ADL reports when the sensing device 104 is plugged in to a power charger, thereby conserving battery power. In some embodiments, ADL sensing device 104 carries out all of the neural network processing and computations in real time, sends ADL reports periodically, but sends emergency reports in real time. In some embodiments, ADL sensing device 104 carries out all of the neural network processing and computations in real time, and sends the ADL reports in real time (e.g., as soon as a report is ready, such as every minute, every five minutes, every twenty minutes, or every hour). In some embodiments, ADL sensing device 104 carries out neural network processing and computations in real time, and sends ADL reports in real time if the sensing device 104 is in communicative range of an intermediary system 106, a monitoring system 120, or other system from which ADL information regarding the user is retrieved. Otherwise, if the sensing device 104 is outside of a communication range (e.g., the user leaves the house and the user's ADL sensing device 104 can no longer wirelessly communicate with the intermediary device 106 or the monitoring system 120), the ADL sensing device 104 continues to sense ADL information and carry out neural network processing, storing ADL reports in local memory (e.g., memory 306) until the sensing device 104 is once again in communication range of an intermediary device 106, monitoring system 120, or other system from which ADL information regarding the user can be retrieved (e.g., the user returns to the house and the user's ADL sensing device 104 sends ADL reports stored in memory 306 to the intermediary device 106 or the monitoring system 120).

Although FIGS. 3C-3D show electronic devices 104-2 and 106-2, FIGS. 3C-3D are intended more as a functional description of the various features which may be present in a user-wearable electronic device and an intermediary device, than as structural schematics of the embodiments described herein. In practice, and as recognized by those of ordinary skill in the art, items shown separately could be combined and some items could be separated.

FIG. 4 is a block diagram illustrating an implementation of a monitoring system 120 (see FIG. 1A) in accordance with some embodiments. In some embodiments, monitoring system 120 includes one or more processors 410, sometimes called CPUs, or hardware processors, or microcontrollers; memory 406; one or more communication interfaces 414 (e.g., a transceiver, and/or a network interface); input/output (I/O) interface 416; and one or more communication buses 408 for interconnecting these components. I/O interface 416 typically includes display 418, which is optionally a touch-screen display. I/O interface 416 optionally includes a keyboard and/or mouse (or other pointing device) 420, and optionally includes a touch-sensitive touchpad 422. In some embodiments, monitoring system 120 is implemented as a server that does not include input/output interface 416, and instead client systems such as mobile device 122 (FIGS. 1A and 5) are used by caregivers and other people to access reports and information stored in monitoring system 120 and to convey commands to monitoring system 120.

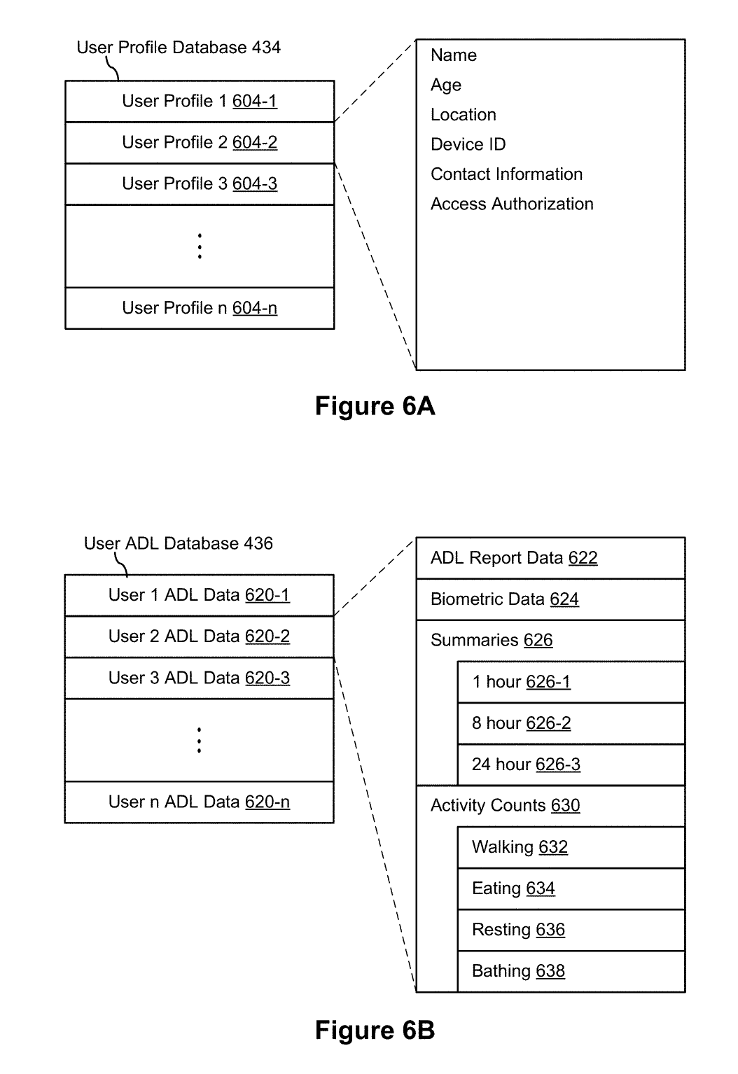

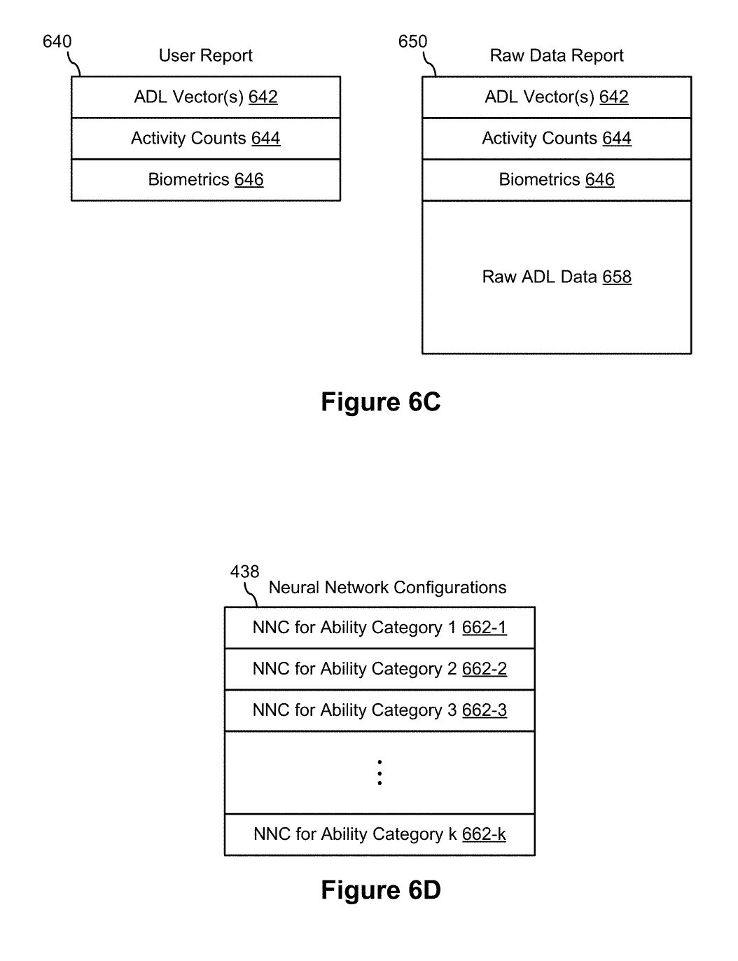

Memory 406 includes high-speed random access memory, such as DRAM, SRAM, DDR RAM or other random access solid state memory devices, and may include non-volatile memory, such as flash memory devices, or other non-volatile solid state storage devices. Memory 406, or alternately the non-volatile memory device(s) within memory 406, comprises a non-transitory computer readable storage medium. In some embodiments, memory 406, or the computer readable storage medium of memory 306 stores the following programs, modules, and data structures, or a subset or superset thereof:
operating system 411, for handling basic computer functions of monitoring system 120;
communications module 412 used for receiving reports and exchanging information with other devices and systems via the one or more communications interfaces 414;
a database or table or other collection of user profiles 434 of users for whom monitoring system 120 monitors activities of daily living and optionally other health or welfare information; user profiles are optionally implemented as or stored in a user profile database, as shown in FIG. 6A;
user ADL database 436, which includes ADL information, and optionally other health and welfare information, received by monitoring system 120 from one or more user-wearable electronic devices 104 (see FIGS. 1A and 2);
neural network configuration(s) 438, which are used to configure the one or more neural networks in ADL monitoring device 104, in intermediary device 106, or in monitoring system 120 itself; the neural network configuration(s) 438 are generated by one or more other systems configured to train one or more similar neural networks using training data, and then stored in memory 406 of monitoring system 120; in embodiments in which neural network processing of raw ADL data is performed by ADL monitoring device 104 or intermediary device 106, respective neural network configurations 438 are transmitted to such systems;
one or more report generation modules 440, which generate reports, described in more detail below, such as status reports regarding the monitored users, and priority reports (e.g., to report emergency situations); in some embodiments report generation module(s) 440 include a priority report generator 442, a system report generator 444, and optionally additional report generators.

In some embodiments, memory 406, or the computer readable storage medium of memory 406 also stores one or more neural networks (e.g., similar to neural networks 316, FIG. 3A or 3D, but not shown in FIG. 4), described in more detail elsewhere in this document, for processing raw ADL data from at least one of the ADL sensors 204 so as to determine which activities of daily living the user wearing the device has been engaged in during each of a sequence of time periods. In such embodiments, raw ADL data from one or more user-wearable ADL sensing devices 104 is transmitted directly or indirectly from such devices 104 to monitoring system 120, and monitoring system 120 processes the raw ADL data from each such user-wearable ADL sensing device 104 using one or more neural networks configured to recognize ADLs corresponding to the user of the user-wearable ADL sensing device 104.

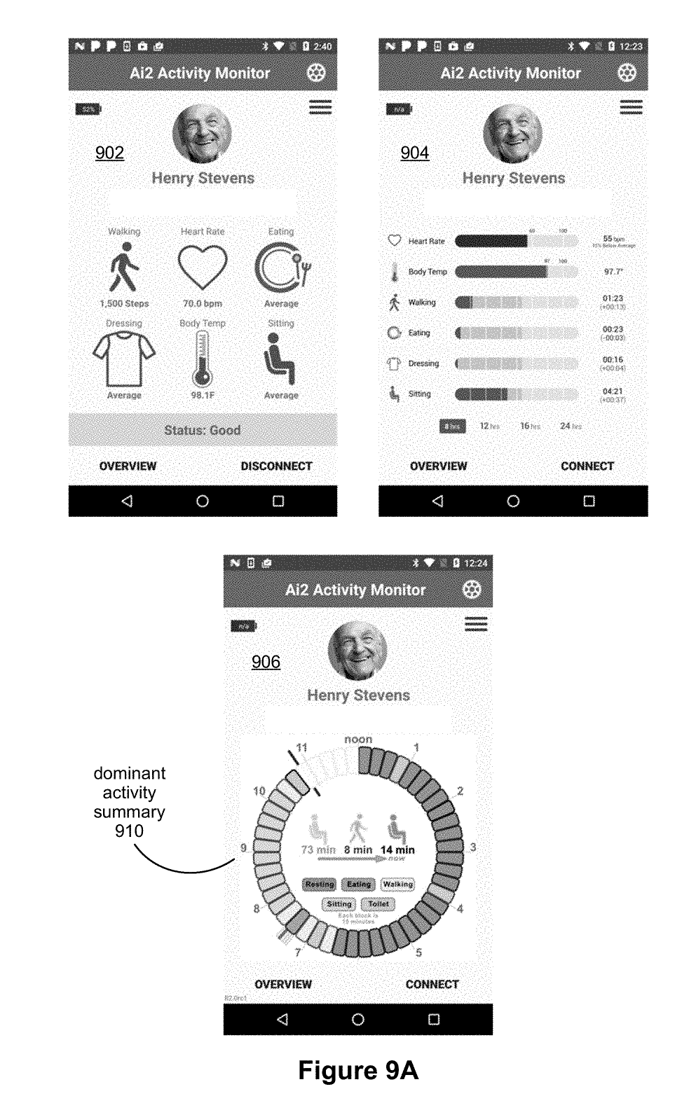

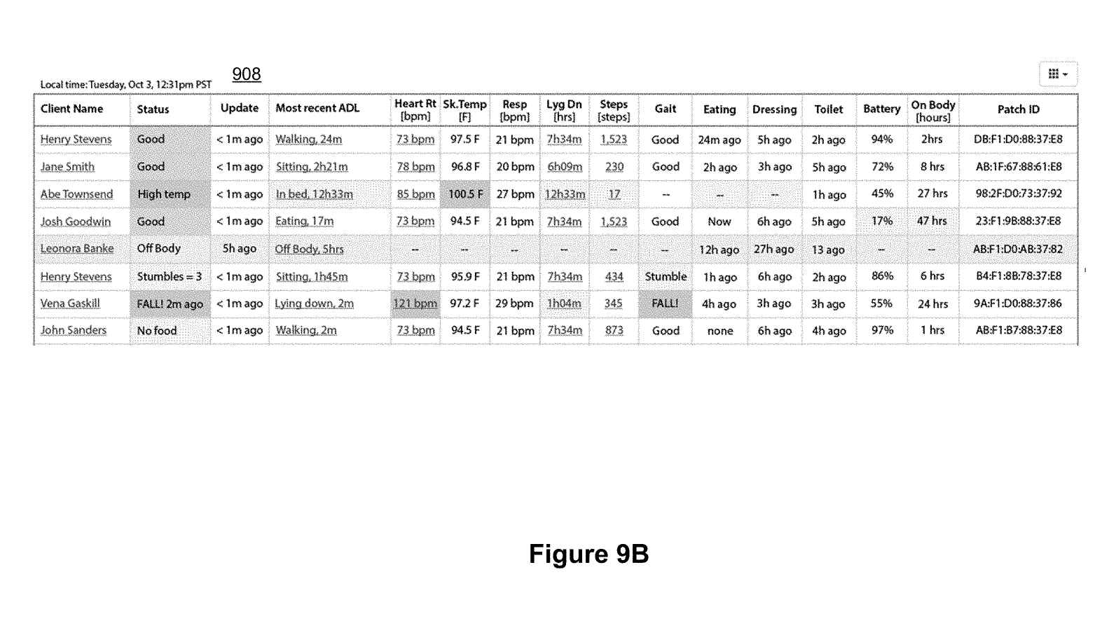

FIGS. 9A and 9B illustrate examples of reports or user interfaces generated by report generation module(s) 440 of monitoring system 120, in accordance with some embodiments. FIG. 9A shows three user interfaces, 902, 904, and 906, for viewing ADL information, and other health and welfare information of a respective monitored user. Interface 906 includes a segmented, clock-shaped dominant activity summary 910, having a sequence of time ordered segments, each representing or providing information identifying a dominant activity of a respective monitored user during a corresponding time period. In this example of interface 906, each segment represents a fifteen minute period, although periods of other durations, such as 5 minutes, 10 minutes, 20 minutes, 30 minutes or 60 minutes, may be used in other embodiments. The physical position of each segment of summary 910 in user interface 906 corresponds to the time period for which that segment provides dominant activity information, for example in accordance with the hour hand position of the segment in a circularly arranged set of positions. In some embodiments, the color of each segment indicates the dominant detected activity of the monitored user during the corresponding time period. In some embodiments, selection of a respective segment, for example by moving a cursor (e.g., using a mouse) or focus selector (e.g., using a touch input on a touch-sensitive display or track pad) over the segment, causes the monitoring system to display additional ADL information for the corresponding time period, for example by showing the dominant activity during each of a sequence of shorter time periods (e.g., 3 minute time periods) within the time period (e.g., a 15 minute time period) corresponding to the selected segment.

FIG. 9B illustrates a dashboard interface 908, providing ADL and optionally other health and welfare information for each of a plurality of monitored users. In some embodiments, dashboard interface 908 highlights information indicative of potential health problems or emergency situations with respect to particular users, and/or provides information to the operator of dashboard interface 908 identifying monitored users for whom additional information meeting predefined criteria (e.g., emergency or potential health problem criteria) is available.

Each of the above identified elements may be stored in one or more of the previously mentioned memory devices that together form memory 406. Each of the above mentioned modules or programs, including the aforementioned modules and operating system, corresponds to a set of instructions and data for performing a function described above. The above identified modules or programs (i.e., sets of instructions) need not be implemented as separate software programs, procedures or modules, and thus various subsets of these modules may be combined or otherwise re-arranged in various embodiments. In some embodiments, memory 406 may store a subset of the modules and data structures identified above. Furthermore, memory 406 may store additional modules and data structures not described above. In some embodiments, the programs, modules, and data structures stored in memory 406, or the computer readable storage medium of memory 406, provide instructions for implementing respective operations of the methods described herein.

Although FIG. 4 shows an electronic monitoring system 120, FIG. 4 is intended more as a functional description of the various features which may be present in a monitoring system, than as a structural schematic of the embodiments described herein. In practice, and as recognized by those of ordinary skill in the art, items shown separately could be combined and some items could be separated.

FIG. 5 is a block diagram illustrating an implementation of a mobile device 122 (see FIG. 1A) in accordance with some embodiments. In some embodiments, mobile device 122 includes one or more processors 510, sometimes called CPUs, or hardware processors, or microcontrollers; memory 506; one or more communication interfaces 514 (e.g., a transceiver, and/or a network interface); input/output (I/O) interface 516; and one or more communication buses 508 for interconnecting these components. I/O interface 516 typically includes a display, which is optionally a touch-screen display. For embodiments in which monitoring system 120 (see FIG. 4) is implemented as a server that does not include input/output interface 416, mobile device 122 functions as a client system and is used by caregivers to access reports and information stored in monitoring system 120 and to convey commands to monitoring system 120. For embodiments in which monitoring system 120 (see FIG. 4) does include input/output interface 416, mobile device 122 functions as an optional, and more mobile, client system that can be used by caregivers in addition, or in the alternative, to monitoring system 120 to access reports and information stored in monitoring system 120 and to convey commands to monitoring system 120.

Memory 506 includes high-speed random access memory, such as DRAM, SRAM, DDR RAM or other random access solid state memory devices, and may include non-volatile memory, such as flash memory devices, or other non-volatile solid state storage devices. Memory 506, or alternately the non-volatile memory device(s) within memory 506, comprises a non-transitory computer readable storage medium. In some embodiments, memory 506, or the computer readable storage medium of memory 506 stores the following programs, modules, and data structures, or a subset or superset thereof:
operating system 511, for handling basic computer functions of mobile device 122;
communications module 512 used for receiving reports and exchanging information with other devices and systems (e.g., monitoring system 120) via the one or more communications interfaces 514;
access rights module 534 used for determining access rights associated with the caregiver designated to use the particular mobile device 122; and
monitoring module 536, which facilitates monitoring and review of ADL information by presenting or configuring for presentation (e.g., on a display included in communications interface 514) ADL information and/or reports received from monitoring system 120, corresponding to one or more user-wearable electronic devices 104 (see FIGS. 1A and 2).

Each of the above identified elements may be stored in one or more of the previously mentioned memory devices that together form memory 506. Each of the above mentioned modules or programs, including the aforementioned operating system, corresponds to a set of instructions and data for performing a function described above. The above identified modules or programs (i.e., sets of instructions) need not be implemented as separate software programs, procedures or modules, and thus various subsets of these modules may be combined or otherwise re-arranged in various embodiments. In some embodiments, memory 506 may store a subset of the modules and data structures identified above. Furthermore, memory 506 may store additional modules and data structures not described above. In some embodiments, the programs, modules, and data structures stored in memory 506, or the computer readable storage medium of memory 506, provide instructions for implementing respective operations of the methods described herein.

Although FIG. 5 shows an electronic mobile device 122, FIG. 5 is intended more as a functional description of the various features which may be present in a mobile device, than as a structural schematic of the embodiments described herein. In practice, and as recognized by those of ordinary skill in the art, items shown separately could be combined and some items could be separated.

FIG. 6A is a block diagram illustrating an implementation of a user profile database 434 in accordance with some embodiments. User profile database 434 includes a set of user profiles 604, for example user profiles 604-1 to 604-n for users 1 to n. In some embodiments, each user profile 604 includes the following information, or a subset or superset thereof: the user's name, age or birthdate, location (e.g., home address or other information identifying the location at which the user lives), an identifier of the user-wearable electronic device 104 used by the user; contact information for the user; and information identifying which people or groups of people are authorized to access the user's ADL information (or more generally ADL, health and welfare information). In some embodiments, a respective user profile includes additional information not listed here. For example, in some embodiments a user may use two user-wearable electronic devices, for example in rotation, with one being worn while the other is recharging, and in such embodiments, the user profile of the user includes device identifiers for both user-wearable electronic devices used by that user. In some embodiments, a respective user profile does not include some of the information items listed here.

FIG. 6B is a block diagram illustrating an implementation of a user ADL database 436 in accordance with some embodiments. User ADL database 436 includes ADL data 620 (e.g., data 620-1 for user 1, through data 620-n for user n), which optionally includes other health and welfare information, for each of a plurality of users. In some embodiments, the ADL data 620 for each respective user includes the following information, or a subset or superset thereof:
ADL report data 622, which includes ADL information included in reports received from the electronic device 104 monitoring the user's ADLs;
biometric data 624, which includes biometric data or information included in reports received from the electronic device 104 monitoring the user's ADLs;
summaries 626, which are summaries or digests of the user's ADL information for various periods of time, such as one hour (e.g., a summary 626-1 of the user's ADL information for a period of one hour, typically the hour immediately preceding the current time), eight hours (e.g., a summary 626-2 of the user's ADL information for a period of eight hours, typically the eight hours immediately preceding the current time), and twenty-four hours (e.g., a summary 626-3 of the user's ADL information for a period of twenty-four hours, typically the twenty-four hours immediately preceding the current time), and/or any period of time corresponding to a caregiver-specific shift (e.g., a summary of the user's ADL information over the course of the caregiver's entire shift); and
activity counts 630, such as a number of times a user performed a first activity 632 (e.g., walking), a second activity 634 (e.g., eating), a third activity 636 (e.g., resting), a fourth activity 638 (e.g., bathing), any subset of activities, and/or any additional activities.

Examples of activity counts 630 include a walking activity count 632, which is or includes, for example, a count of steps by the user, or a count of minutes in which the user was walking, during one or more predefined periods of times, such as fifteen minutes, one hour, eight hours, and/or twenty-four hours; an eating activity count 634, which is or includes a count of minutes in which the user was eating, during one or more predefined period of times, such as fifteen minutes, one hour, eight hours, and/or twenty-four hours; a resting activity count 636, which is or includes, for example, a count of minutes in which the user was resting (remaining stationary, or napping, or sleeping), during one or more predefined period of times, such as fifteen minutes, one hour, eight hours, and/or twenty-four hours; and/or a bathing activity count 638, which is or includes, for example, a count of minutes in which the user was bathing or engaged in hygiene activities, during one or more predefined period of times, such as fifteen minutes, one hour, eight hours, and/or twenty-four hours. Another example of an activity count that is optionally determined by device 104 for each of one or more respective time periods, and included in activity counts 630, is a count of the number of times the user has tripped or stumbled. In the aforementioned examples, each count of the number of instances of an ADL being performed may be considered with a corresponding count of time during which the instances were being performed in order to calculate a health-related value. For example, a user who walks for S minutes during a given period of time will have a higher level of health, welfare, fitness, and/or activity than a user who walks for T minutes during the given period of time, where T is less than S (sometimes represented as T<S).

FIG. 6C is a block diagram illustrating information included in a user report 640 and information included in a raw data report 650, in accordance with some embodiments. User reports 640 and raw data reports 650 are reports generated by a respective user-wearable electronic device 104 and sent to monitoring system 120. In some embodiments, user reports 640 are generated by device 104 at evenly spaced reporting intervals, such as fifteen minutes, and include information for a corresponding report period. In some embodiments, a respective user report 640 includes ADL vectors 642 (described in more detail below) for the report period, activity counts 644 (e.g., activity counts for activities such as walking, eating, hygiene and resting) for the report period, and biometrics 646 (e.g., temperature and heart rate) for the report period.

In some embodiments, user-wearable electronic device 104 generates raw data reports 650 so as to provide monitoring system 120, or one or more other systems, with raw ADL data 658 to enable the generation of improved, or personalized, neural network configurations. In some embodiments, a respective raw data report 650 includes ADL vectors 642 (described in more detail below) for a report period, activity counts 644 (e.g., activity counts for activities such as walking, eating, hygiene and resting) for the report period, biometrics 646 (e.g., temperature and heart rate) for the report period, and raw ADL data 658 for the report period.

FIG. 6D is a block diagram illustrating neural network configurations 438 in accordance with some embodiments. In some embodiments, user-device 104 is configured to detect ability-specific activities which differ according to particular ability categories. Accordingly, memory 406 includes neural network configurations (NNCs) 662 programmed for detecting ADLs specific to different ability levels (e.g., 662-1 for NNC for ability category 1 through 662-k for NNC for ability category k). In some embodiments, an ability category includes a predefined set of ADLs that includes one or more, two or more, or three or more ADLs specific to the ability category, and optionally includes one or more generic ADLs common to multiple ability categories.

Exemplary ability categories in accordance with some embodiments include, but are not limited to: ambulation-impaired activities (e.g., for users who use a cane, walker, or wheelchair for ambulation), and motor function-impaired activities (e.g., for users who have difficulty moving their hands, arms, hips, and the like). ADLs in different ability categories are detected using category-specific NNCs. For example, wheelchair-based ambulation is associated with different torso movements than walking-based ambulation, and accordingly, a first NNC is used for detecting ambulation based on movements associated with operating a wheelchair, and a second (different from the first) NNC is used for detecting ambulation based on movements associated with walking. As a further example, a motor function-impaired person may bathe by sitting in a bath, while a non-impaired person may bathe by standing in a shower. Accordingly, a first NNC is used for detecting bathing based on movements associated with taking a bath, and a second (different from the first) NNC is used for detecting bathing based on movements associated with taking a shower.

In some embodiments, memory 406 initially includes a generic ability category NNC, which enables device 104 to be used without a preprogrammed ability-specific NNC. In some embodiments, the generic NNC is subsequently updated or replaced with an updated neural network configuration according to a received update or based on subsequent training, resulting in processor(s) 210 reconfiguring or replacing the generic NNC with the updated configuration, thereby enabling ability-specific ADL identification information for time periods subsequent to the reconfiguring of the ADL sensing device 104 with an ability-specific NNC.

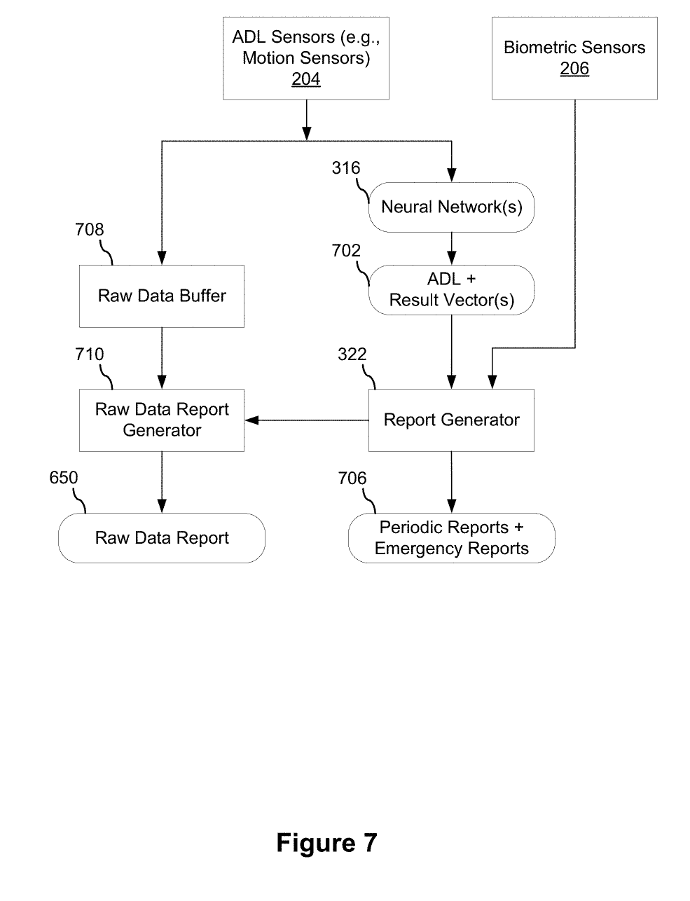

FIG. 7 is a flow chart illustrating data flow in an implementation of a user-wearable electronic device 104, in accordance with some embodiments. As shown, information from one or more ADL sensors 204, for example a motion sensor (e.g., an accelerometer), is provided to one or more pre-trained neural networks 316, which produce one or more result vectors 702 for each report period. For example, in some embodiments, pre-trained neural networks 316 generate a set of result vectors every six seconds, and the result vectors for a report period, such as fifteen minutes are combined or collected by report generation module(s) 322, which then produces a digest or other report 706 for each time period, sometimes called a periodic report (e.g., periodic user reports 640, FIG. 6C) for transmission to monitoring system 120. In some embodiments, reports 706 are transmitted at a predefined time of day or night, at a predefined time relative to a start or end time of a caregiver's shift, in response to an emergency (e.g., a detected fall), and/or on demand (e.g., upon request by a caregiver operating mobile device 122 or monitoring system 120). In some embodiments, the result vectors include information useable to produce activity counts, such as the activity counts discussed elsewhere in this document. In some embodiments, report generation module(s) 322 also produces emergency reports 706 when the result vectors it receives from neural networks 316, and/or the biometric information it receives from biometric sensors 206, satisfy emergency report generation criteria. Examples of emergency report generation criteria are discussed below.

In some embodiments, raw ADL sensor data is temporarily stored in a raw data buffer 708 in user-wearable electronic device 104, which, along with the report data included in the aforementioned periodic reports is provided to a raw data report generator 710, which produces a raw data report (e.g., raw data report 650, FIG. 6C) for transmission to monitoring system 120.

Through the use of one or more trained neural networks 316 in user-wearable electronic device 104, ADLs are associated with certain characteristic motions and/or orientations. As a nonlimiting example, eating is typically associated with a forward-leaning motion or similar torso motion as the user inserts a utensil with food into the user's mouth. Similarly, other ADLs are associated with other patterns of movement and/or orientation. One or more neural networks in user-wearable electronic device 104 are trained to recognize motion and/or orientation patterns consistent with eating, and each of the other ADLs that device 104 is configured to monitor.

In some embodiments, as shown in FIGS. 2 and 3, at least one processor 210 is coupled to the aforementioned sensors, and receives raw sensor data from ADL sensor 204 (hereinafter “raw ADL data”) and raw sensor data from biometric sensor 206 (hereinafter “raw biometric data”). In some embodiments, processor 210 receives the raw ADL data and/or the raw biometric data at a rate of no less than 10 samples per second, in accordance with a sampling period.

For each time period in a sequence of successive time periods, processor 210 generates ADL identification information for the time period by processing the raw ADL data produced by ADL sensor 204 using one or more neural networks pre-trained to recognize a predefined set of ADLs. In some embodiments, the successive time periods each have a duration of no more than 30 seconds (for example, 6 seconds). In some embodiments, processor 210 processes at least 10 samples of raw ADL data for each time period of the successive time periods. Further, in some embodiments, a ratio of the time period (at which processor 210 generates ADL identification information) to the sampling period (at which processor 210 samples raw data) is no less than 100, and is typically between 100 and 5,000. In some embodiments, each pre-trained neural network includes a plurality of neural network layers, and at least one layer of the plurality of neural network layers is, or includes, a recurrent neural network. An output of the neural network for each time period corresponds to the generated ADL identification information for the respective time period.

In some embodiments, processor 210 generates the ADL identification information for a respective time period in the sequence of time periods by generating a set of scores, including one or more scores for each ADL in the predefined set of ADLs. In accordance with the generated set of scores, processor 210 determines a dominant activity for the respective time period, wherein the dominant activity is one of the ADLs in the predefined set of ADLs. In accordance with a determination that the one or more scores for the dominant activity for the respective time period meets predefined criteria, processor 210 includes in the generated ADL identification information for the respective time period information identifying the dominant activity for the respective time period. However, in accordance with a determination that the one or more scores for the dominant activity for the respective time period do not meet the predefined criteria, processor 210 includes in the generated ADL identification information for the respective time period information indicating that the user's activity during the respective time period has not been classified as any of the ADLs in the predefined set of ADLs. In some embodiments, the predefined set of ADLs includes N distinct ADLs, where N is an integer greater than 2, and the ADL identification information generated by the one or more processors for the time period includes a vector of having at least N+1 elements, only one of which is set to a non-null value. In other embodiments, the predefined set of ADLs includes N distinct ADLs, where N is an integer greater than 2, and the ADL identification information generated by the one or more processors for the time period includes a vector of having at least N elements, only one of which is set to a non-null value.

In some embodiments, proximity receiver 212 is disposed in or on housing 202. Proximity receiver 212 obtains location or proximity information (hereinafter, “raw proximity information”) corresponding to a range or proximity to one or more beacons 132-138 (see FIG. 1B) at known locations in an environment occupied by the user, and communicates the raw proximity information to processor 210, which determines location information of the user based on the raw proximity information. In some embodiments, location information includes a room in which the user is located (e.g., bedroom, bathroom, kitchen, or any other living room). In some embodiments, processor 210 uses the location information to supplement the raw ADL data in order to more accurately generate ADL identification information. For example, by taking advantage of certain location-based ADL assumptions (e.g., a person does not bathe in the kitchen, and likewise, does not eat in the bathroom), the one or more neural networks narrow down a subset of possible ADL identification information for a given set of raw ADL data.

In some embodiments, transceiver 214 is disposed in housing 202 and coupled to processor 210. Transceiver 214 obtains ADL identification information for a sequence of time periods from processor 210, and transmits reports for the user. In some embodiments, transceiver 214 transmits the reports at predefined times at intervals of no less than 5 minutes (for example, fifteen minutes). In other embodiments, transceiver 214 transmits the reports only when device 104 is connected to an external power source or otherwise receiving power from an external power source, for example so as to charge the internal battery 216 of the device. Further, in some embodiments, transceiver 214 transmits the reports in response to a manual transmission command (e.g., by pressing a “transmit” button on device 104, or by a caregiver requesting the reports while using monitoring system 120 or mobile device 122). In some embodiments, reports are transmitted at a predetermined transmission rate (e.g., every fifteen minutes, every hour, every four hours, every eight hours, and/or once a day), but with aggregated ADL identification information from a plurality of time periods (e.g., ADL counts for five-minute or fifteen-minute windows of time). It is understood that the aforementioned reporting times and aggregation periods are exemplary, and a person of ordinary skill in the art may configure them to be set in accordance with caregiver-determined requirements and/or user-specific needs. In some embodiments, a respective report includes ADL information (e.g., a list of ADLs detected during given time periods) corresponding to the generated ADL identification information for one or more time periods in the sequence of time periods. In some embodiments, in addition to including ADL information, a respective report also includes biometric information corresponding to the raw biometric data generated by the biometric sensor for a time period corresponding to the respective report.

Further, in some embodiments, in addition to or as an alternative to including ADL identification information, a respective report (e.g., raw data report 650, FIG. 6C) includes raw ADL data that has been stored by processor 210. While processor 210 temporarily stores raw ADL data for one or more time periods, in some embodiments, the raw ADL data is not transmitted to a target system until device 104 is connected to an external power source (e.g., plugged into a power charger), in order to save battery power. Raw ADL data may be transmitted for use in the development of new or improved neural network configurations in order to, for example, identify additional classifications of activity, or improve the classification of raw ADL data into the predefined set of ADLs or other predefined categories (e.g., tripping, stumbling, exercise, etc.). In some embodiments, raw ADL data transmissions for the aforementioned purposes may be prompted by the determination that the one or more scores for the dominant activity for the respective time period do not meet the predefined criteria, as disclosed above. In other words, if raw ADL data cannot be classified as a particular ADL, or the classification has a low confidence score or other indicia of not meeting predefined reliability criteria, processor 210 stores the raw ADL data and transmits it for further analysis. In some embodiments, other data is transmitted along with the raw ADL data (e.g., the ADL identification vector(s), the biometric data, and/or the scores generated using the raw ADL data for the respective time periods).

In some embodiments, processor 210 automatically detects an emergency, based on the raw ADL data and/or biometric data, in accordance with predefined emergency detection criteria. In response to the automatic detection of an emergency, processor 210 initiates transmission of an emergency report to the target system using transceiver 214. In some embodiments, the criteria for identifying an emergency include one or more of: heart rate high or low (e.g., above a first heart rate threshold or below a second heart rate threshold); body temperature high or low (e.g., above a first temperature threshold or below a second temperature threshold); breathing rate high or low (e.g., above a first breathing rate threshold or below a second breathing rate threshold); falling or stumbling; not sleeping during a time period of at least a predefined minimum duration; not eating or drinking during a time period of at least a predefined minimum duration; coughing in excess of a predefined threshold; choking; a crossed threshold of time during which an activity has been performed (e.g., sleeping or remaining in one position for too long); a crossed threshold of time between particular activities (e.g., too much time between meals); a crossed threshold of activity counts (e.g., too many or too little ADL events compared to a predefined goal or standard); or a combination of such factors.

In some embodiments, and with reference to FIG. 1A, transceiver 214 wirelessly transmits the reports for the user to an intermediary device 106, which forwards the reports for the user to a target system (e.g., monitoring system 120 or mobile device 122). In some embodiments, intermediary device 106 is a power charger for device 104, while in other embodiments, intermediary device 106 is a second instance of device 104 located in the same environment as the user (e.g. in the same building as, or otherwise co-located with, the user-wearable electronic device 104). In some embodiments, intermediary device 106 is a docking station which serves as a repository for a user to return his or her device 104, where the device 104 recharges and transmits data that may not have been otherwise transmitted while being worn by the user. In such embodiments, while the device 104 is being recharged (e.g., in a docking station), a replacement, backup, or alternative device 104 may be worn by the user so that the user's ADLs are always being monitored.

In some embodiments, transceiver 214 receives an updated configuration for the one or more neural networks, and sends the updated configuration to processor 210, which reconfigures the one or more neural networks with the updated configuration. As a result, processor 210 thereafter generates ADL identification information for subsequent time periods using the one or more neural networks with the updated configuration. In some embodiments, all of the one or more neural networks are updated with new configurations at the same time. In some embodiments, or in some circumstances, just one of the neural networks is updated with a new configuration, or a subset of the neural networks are updated with new configurations when one or more updated configurations are received by device 104. In some embodiments, the updated configuration allows for more accurate user-specific ADL identification schemes, based on analysis of previously received raw ADL data. For example, the updated configuration allows for personally targeted ADL identification schemes based on the user's personal habits (e.g., washing hair in the sink versus in the shower) or the user's historical activities (e.g., dancing, petting animals, etc.). In some embodiments, the updated configuration is customized in accordance with the geographic region of the user or cultural or other classifications associated with the user, or a combination thereof. In some embodiments, transceiver 214 is a wireless transceiver, while in other embodiments, transceiver 214 is a wired transceiver.

In some embodiments, rechargeable battery 216 is disposed within the housing, and processor 210 performs a predefined set of tasks while device 104 is determined to be connected to an external power source for recharging the battery. In some embodiments, the predefined set of tasks includes transmitting (e.g., through transceiver 214) recorded information that was not transmitted while the system was not connected to the external power source. Further, in some embodiments, the predefined set of tasks includes receiving (e.g., through transceiver 214) update information for reconfiguring at least one aspect of device 104 (e.g., an updated configuration for the one or more neural networks as disclosed above).

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A user-wearable electronic device, for monitoring user activities of daily living (ADL), comprising:
a housing configured to be worn on a user's torso;
a plurality of sensors disposed in the housing, including a first sensor to produce raw ADL data, and a biometric sensor configured to sense one or more biometric characteristics of the user and generate corresponding biometric data;
one or more processors, disposed in the housing and coupled to the one or more sensors, configured to:
for each time period in a sequence of successive time periods,
generate ADL identification information for the time period by processing the raw ADL data produced by the first sensor using one or more neural networks pre-trained to recognize a predefined set of ADLs, the pre-trained one or more neural networks each including a plurality of neural network layers, at least one layer of the plurality of neural network layers comprising a recurrent neural network, wherein an output of the one or more neural networks for each time period corresponds to the generated ADL identification information for the time period; and
a transmitter, disposed in the housing and coupled to at least one of the one or more processors, to transmit, at predefined times, reports for the user, wherein a respective report for the user includes ADL information corresponding to the generated ADL identification information for one or more time periods in the sequence of time periods.

2. The user-wearable device of claim 1, wherein the biometric sensor and housing are configured to operatively couple the biometric sensor to the user's skin.

3. The user-wearable device of claim 1, wherein the housing includes an interface configured to be in direct contact with the user's skin, and the biometric sensor is coupled to the interface.

4. The user-wearable device of claim 1, wherein the respective report for the user further includes biometric information corresponding to the biometric data generated by the biometric sensor for a time period corresponding to the respective report.

5. The user-wearable device of claim 1, wherein the biometric sensor includes at least one of: a temperature sensor for sensing temperature of the user, an EKG sensor for detecting electrical activity associated with the user's heart, a heart rate sensor for measuring the user's heart rate, and a blood pressure sensor for measuring at least one parameter of the user's blood pressure.

6. The user-wearable device of claim 1, wherein the transmitter is configured to transmit the reports to a remotely located monitoring system that monitors ADL information for a plurality of users.

7. The user-wearable device of claim 1, wherein the predefined set of ADLs includes three or more activities selected from the group consisting of dressing, eating, transferring/ambulation, continence/toileting, bathing/hygiene, sitting, and sleeping/napping.

8. The user-wearable device of claim 7, wherein the predefined set of ADLs further includes one or more activities selected from the group consisting of shopping, housekeeping, meal preparation, transportation, and talking/socializing/communication.

9. The user-wearable device of claim 1, wherein the first sensor comprises an accelerometer, an orientation sensor, motion sensor, or gyroscopic sensor.

10. The user-wearable device of claim 1, further comprising a location or proximity sensor disposed in or on the housing;
wherein the one or more processors are further configured to:
determine location information for the user based on data from the location or proximity sensor; and
generate at least a portion of the ADL identification information for the time period by processing the raw ADL data produced by the first sensor and the location information for the user using at least one of the one or more neural networks.

11. The user-wearable device of claim 10, wherein the location or proximity sensor is configured to obtain or generate range or proximity information corresponding to a range or proximity to one or more beacons at known locations in an environment occupied by the user; and wherein at least one processor of the one or more processors is configured to determine the location information for the user based on the range or proximity information.

12. The user-wearable device of claim 1, wherein the transmitter is configured to wirelessly transmit the reports for the user to an intermediary device co-located with the user-wearable device that forwards the reports for the user to a target system.

13. The user-wearable device of claim 1, wherein the one or more processors is configured to generate the ADL identification information for a respective time period in the sequence of time periods by:
generating a set of scores, including one or more scores for each ADL in the predefined set of ADLs;
in accordance with the generated set of scores, determining a dominant activity for the respective time period, wherein the dominant activity is one of the ADLs in the predefined set of ADLs;
in accordance with a determination that the one or more scores for the dominant activity for the respective time period meets predefined criteria, including in the generated ADL identification information for the respective time period information identifying the dominant activity for the respective time period.

14. The user-wearable device of claim 13, wherein the one or more processors are further configured to generate the ADL identification information for a respective time period in the sequence of time periods by:
in accordance with a determination that the one or more scores for the dominant activity for the respective time period do not meet the predefined criteria, including in the generated ADL identification information for the respective time period information indicating that the user's activity during the respective time period has not been classified as any of the ADLs in the predefined set of ADLs.

15. The user-wearable device of claim 1, wherein the predefined set of ADLs includes N distinct ADLs, where N is an integer greater than 2, and the ADL identification information generated by the one or more processors for the time period includes a vector of having at least N+1 elements, only one of which is set to a non-null value.

16. The user-wearable device of claim 1, wherein the predefined set of ADLs includes N distinct ADLs, where N is an integer greater than 2, and the ADL identification information generated by the one or more processors for the time period includes a vector of having at least N elements, only one of which is set to a non-null value.

17. The user-wearable device of claim 1, wherein
the pre-trained one or more neural networks include a first neural network having a first configuration;
the user-wearable device includes a receiver, disposed in the housing and coupled to at least one processor of the one or more processors, to receive an updated configuration for the first neural network; and
the one or more processors are further configured to reconfigure the first neural network with the updated configuration, and to thereafter generate ADL identification information for time periods subsequent to the reconfiguring of the first neural network, using the first neural network configured using the updated configuration.

18. The user-wearable device of claim 17, wherein the transmitter and the receiver comprise a wireless transceiver.

19. The user-wearable device of claim 1, wherein the housing has a length no greater than 10 cm, a width no greater than 6.5 cm and a thickness no greater than 0.5 cm.

20. The user-wearable device of claim 1, wherein the housing and all components within the housing have a total weight no greater than 60 grams.

21. The user-wearable device of claim 1, wherein the successive time periods each have a duration of no more than 30 seconds, and the predefined times at which the transmitter transmits reports for the user occur at intervals of no less than 5 minutes.

22. The user-wearable device of claim 1, wherein the one or more processors are configured to receive raw ADL data from the first sensor at a rate of no less than 10 samples per second, in accordance with a sampling period, and a ratio of the time period to the sampling period is no less than 100.

23. The user-wearable device of claim 1, wherein the one or more processors are configured to store the raw ADL data for one or more of the time periods in the sequence of time period, and to initiate transmission of the stored raw ADL data to the target system using the transmitter.

24. The user-wearable device of claim 1, wherein the one or more processors are further configured to: automatically detect an emergency, based on the raw ADL data and/or biometric data, in accordance with predefined emergency detection criteria, and in response to the automatic detection of the emergency, to initiate transmission of an emergency report to the target system using the transmitter.

25. The user-wearable device of claim 1, further comprising a rechargeable battery disposed within the housing, wherein the one or more processors are further configured to:
perform a predefined set of tasks while the user-wearable device is determined to be connected to a power source for recharging the user-wearable device's battery, the predefined set of tasks including transmitting recorded information not transmitted when the user-wearable device is connected to a power source for recharging the user-wearable device's battery, and receiving update information for reconfiguring at least one aspect of the user-wearable device.

26. A server system, comprising:
memory storing a plurality of neural network configurations, each neural network configurations corresponding to a distinct classification or a distinct group of classifications;
one or more processors for executing one or more programs; and
a communication interface, coupled to at least one of the one or more processors to provide each of the neural network configurations to corresponding user-wearable electronic devices or intermediary devices, each user-wearable electronic device or intermediary device for identifying user activities of daily living (ADL) of a respective user assigned to a classification or group of classifications corresponding to the neural network configuration provided to the user-wearable electronic device or intermediary device.

27. The server system of claim 26, wherein a respective user-wearable electronic device of the corresponding user-wearable electronic devices or a respective intermediary device of the intermediary devices includes:
one or more neural networks configured with the neural network configuration provided by the server system to generate, for each time period in a sequence of successive time periods, ADL identification information for the time period by processing raw ADL data produced by a first sensor using one or more neural networks pre-trained to recognize a predefined set of ADLs, the one or more neural networks each including a plurality of neural network layers, at least one layer of the plurality of neural network layers comprising a recurrent neural network, wherein an output of the one or more neural networks for each time period corresponds to the generated ADL identification information for the time period.

28. An activities of daily living (ADL) monitoring system, comprising:
one or more processors, configured to:
collect raw ADL data produced by a first sensor;
for each time period in a sequence of successive time periods, generate ADL identification information for the time period by processing the raw ADL data produced by the first sensor using one or more neural networks pre-trained to recognize a predefined set of ADLs, the pre-trained one or more neural networks each including a plurality of neural network layers, at least one layer of the plurality of neural network layers comprising a recurrent neural network, wherein an output of the one or more neural networks for each time period corresponds to the generated ADL identification information for the time period; and
transmit one or more reports corresponding to the user, wherein a respective report corresponding to the user includes ADL information corresponding to the generated ADL identification information for one or more time periods in the sequence of time periods.

29. The ADL monitoring system of claim 28, wherein
the system includes a user-wearable electronic device and an intermediary device configured to receive raw ADL from the user-wearable electronic device;
the first sensor is disposed in a housing, located in the user-wearable electronic device, the housing configured to be worn by or affixed to a device worn by a user; and
the ADL identification information is generated by one or more processors in the intermediary device using the one or more neural networks pre-trained to recognize a predefined set of ADLs.

30. The ADL monitoring system of claim 28, wherein the predefined set of ADLs includes three or more activities selected from the group consisting of dressing, eating, transferring/ambulation, continence/toileting, bathing/hygiene, sitting, and sleeping/napping.
